(19) United States
(12) Patent Application Publication
(10) Pub. No.: US 2006/0241593 A1
(43) Pub. Date: Oct. 26, 2006

(54) Multi-piece vertebral attachment device

(75) Inventors: Michael C. Sherman, Memphis, TN (US); Fred J. Molz IV, Collierville, TN (US)

Correspondence Address: COATS & BENNETT, PLLC, P O BOX 5, RALEIGH, NC, 27602, US

(73) Assignee: SDGI Holdings, Inc.

(21) Appl. No.: 11/101,917

(22) Filed: Apr. 8, 2005

(51) Int. Cl. A61F 2/30

(52) U.S. Cl. 606/61

(57) ABSTRACT

A multi-piece vertebral attachment device with a first member, one or more second members, and optionally, a removable plug. The first member may have a fixed, threaded exterior surface to engage a vertebral member. The first member may further include a hollow, threaded interior. The hollow interior may be positioned inside the threaded exterior surface and at least partly positioned within the vertebral member when the first member is inserted in the vertebral member. The second member may have an outer diameter sized to fit within the hollow interior of the first member. A spinal implant device may be coupled to the second member. The second member may be installed during a separate surgical procedure from that of the first. The removable plug may be installed in the first member until the second member is installed. Revision may be performed by removing the second member and inserting a third member.

### BACKGROUND

Spinal implants are used for correction and stabilization of the spine. Such implants often comprise screws engaged with the vertebral bodies and configured for attachment to elongated rods or plates that extend along the vertebral bodies. Thus, the spinal implant components work in concert to provide reconstructive or corrective support for the spine. Because the spine is a flexible, load-bearing structure, the loads imparted on and by the spine can be substantial.

The structural loads that can be applied to spinal implants may be limited by the quality of the interface between the implant and the spine. For example, the ability of the implant to receive applied loads may be limited by poor engagement to individual vertebral bodies. In such cases, the applied corrective load may cause movement of the device relative to the vertebra and the resulting loss of engagement between the implant and the vertebral body. Alternatively, with knowledge of the limited load-bearing capability of conventional bone-implant interfaces, surgeons may opt to limit the corrective load applied during each surgical procedure.

One potential source of this problem results when the interface between vertebral screws and vertebral bodies begins to experience stresses almost immediately following surgery. Surgeons may impose rest and external bracing during post-operation recovery times, but the interface may still be prone to movement. This movement consequently inhibits bone-to-hardware adhesion and bone growth. This, in turn, limits the load bearing capacity at the implant interface.

Another problem arises when revision or multi-stage surgical procedures are performed. In these procedures, vertebral screws may be replaced at some time after the initial installation procedure. Removing the original screws leaves a void in the vertebral member that can limit the holding capability of replacement screws. In any event, the interface between vertebral screws and the vertebral members presents a limiting factor in establishing a structurally solid anchor point for spinal implants.

### SUMMARY

Embodiments of the present invention are directed to a multi-component device to attach to a vertebral member. A first anchor member may have a threaded exterior surface adapted for insertion into and engagement with a vertebral member. The first member may also have a hollow interior with a threaded interior surface. A second attachment member may have an outer diameter sized to fit within the hollow interior of the first member. The second member may also have external threads to mate with a threaded interior surface of the first member. The second member may further be sized to prevent the first member from expanding during insertion of the second member into the first member. The second member may also be adapted to couple to a spinal implant device such as a plate or rod. A removable plug may be inserted into the hollow interior until a time when the second member is to be inserted into the first member.

In use, the device may be attached to a vertebral member by initially inserting the first member into a vertebral member. This first member may be inserted during a first surgical procedure. After a predetermined condition is satisfied to allow the first member to become set within the vertebral member, the second attachment member may be inserted into the first member. Thus, the second member may be installed during a separate surgical procedure. The second member may be inserted to a depth within the vertebral member as to bring a head portion of the attachment member to a working height near the first member. A spinal implant device may then be coupled to the second member. Prior to inserting the second member, a removable plug may be removed from the interior of the first member.

Revision surgery or additional spinal adjustments may be performed during subsequent procedures where the second member may be removed from the first member and replaced with a third member, which may have a different attachment mechanism for coupling to a spinal implant device.

### BRIEF DESCRIPTION OF THE DRAWINGS

FIG. 1 is a superior or inferior profile of an exemplary spinal vertebral member and an uninstalled pedicle attachment device according to one embodiment of the present invention;

FIG. 2 is a superior or inferior profile of an exemplary spinal vertebral member and a partially installed pedicle attachment device according to one embodiment of the present invention;

FIG. 3 is a superior or inferior profile of an exemplary spinal vertebral member and an installed pedicle attachment device according to one embodiment of the present invention;

FIG. 4 is a cross section view of an exemplary anchor member and removable plug according to one embodiment of the present invention;

FIG. 5 is a top axial view of an exemplary anchor member and removable plug according to one embodiment of the present invention;

FIG. 6 is a superior or inferior profile of an exemplary spinal vertebral member and a partially installed pedicle attachment device according to one embodiment of the present invention;

FIG. 7 is a side view of an exemplary anchor member and interchangeable attachment members according to one embodiment of the present invention; and

FIG. 8 is a superior or inferior profile of an exemplary spinal vertebral member and an installed anterior attachment device according to one embodiment of the present invention.

### DETAILED DESCRIPTION

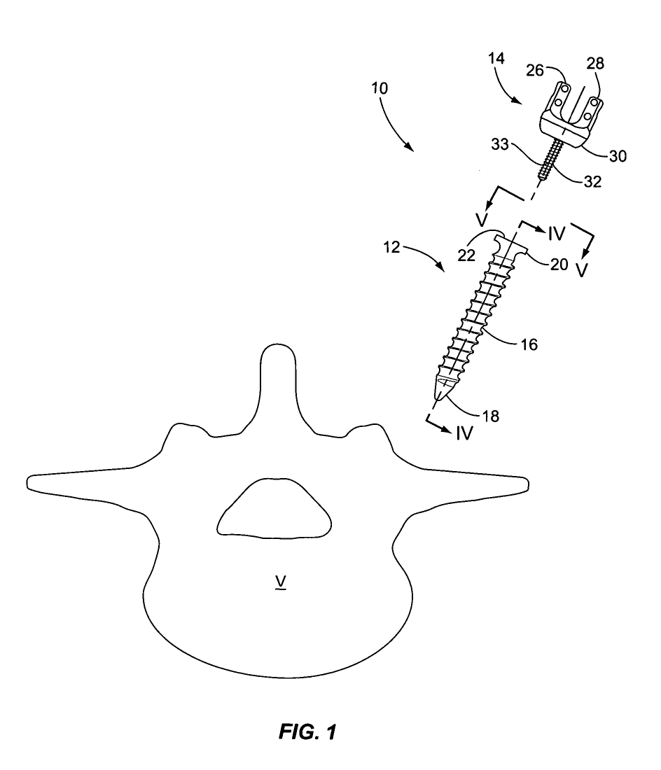

Various embodiments disclosed herein relate to the attachment of spinal implant devices to vertebral members for correcting or treating spinal deformities and conditions. The devices and methods disclosed include multiple components, but may be advantageously configured to attach to conventional spinal implant devices such as rods, plates, and the like. Referring to FIG. 1, a representative attachment device, indicated by the number 10, is shown relative to an outline of an exemplary vertebral element, labeled V. In the embodiment shown, the attachment device 10 comprises an anchor member 12 and an attachment member 14. When combined, the exemplary anchor member 12 and attachment member 14 function as a pedicle screw attachment device for coupling the vertebral element V to a rod member or other spinal implant (not shown in FIG. 1). The anchor member 12 and attachment member 14 may be inserted in a patient during a single surgical implant procedure. However, it is contemplated that the anchor member 12 and the attachment member 14 are installed during separate, temporally-distanced procedures. Thus, the anchor member 12 may be allowed to integrate with the bony structure of vertebral member V over time. Then, the attachment member 14 and other portions of a spinal implant device (not shown) may be installed during a second procedure.

The anchor member 12 may thus be configured with an engagement portion 16 comprising bone threads, knurls, ridges, or other engagement features. In one embodiment, the engagement portion 16 includes threads as are conventionally found in pedicle or other vertebral screws. Anchor member 12 may be constructed of a non-resorbable, biocompatible material, such as carbon-reinforced polymer composites, shape-memory alloys, titanium, titanium alloys, cobalt chrome alloys, stainless steel, ceramics and combinations thereof.

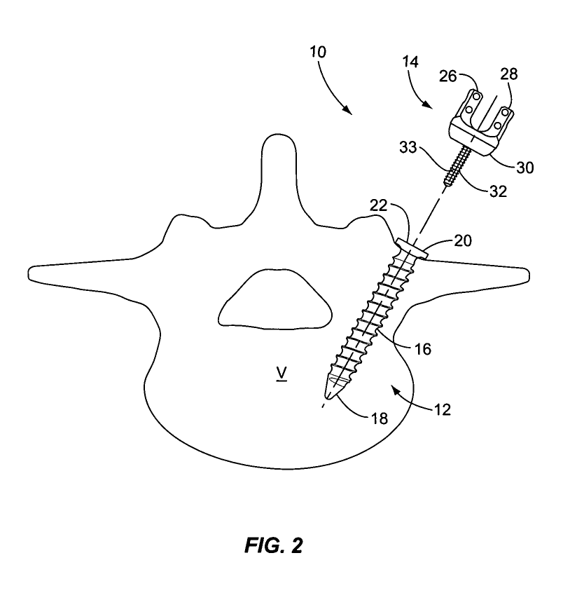

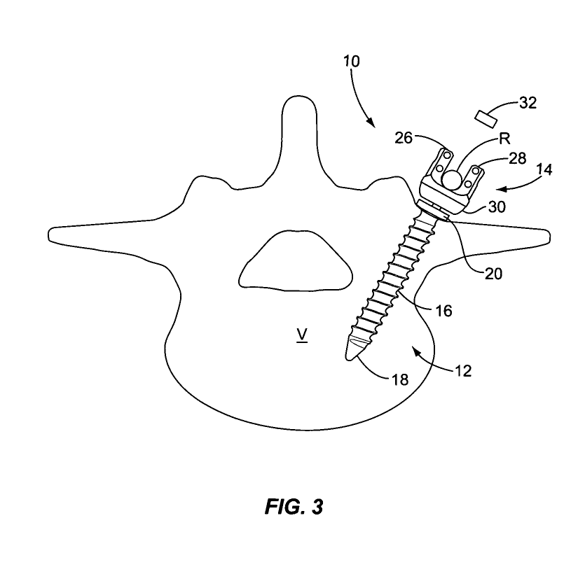

A distal end 18 of the anchor member 12 may be tapered to promote entry of the anchor member 12 into the vertebral member V as shown in FIG. 2. FIG. 2 shows the anchor member 12 installed in the representative vertebral member V. The installation depth of the anchor member 12 within the vertebral member V may be limited by a flange 22 at a proximal end 20 of the anchor member 12. The flange 22 may have a larger cross-section diameter than the engagement portion 16, and thus operates as stop to prevent further entry of the anchor member 12 into the vertebral body V. Once the anchor member 12 is installed (as shown in FIG. 2), the attachment member 14 may be installed (as shown in FIG. 3). That is, the attachment member 14 may be installed during the same surgical procedure or, during a second procedure.

For the second surgical procedure, it is contemplated that anchor member 12 will have integrated with the bony or tissue structure of the vertebral element V, and can have sufficient load carrying capabilities to withstand loading to correct or treat a spinal deformity or condition associated with the spinal column. Thus, the anchor member 12 may be subjected to external loading in a second surgical procedure that can be greater than the loading that could be applied pre-integration. Since the integrated anchor member 12 can be subjected to higher initial loading, the desired surgical result may be achieved more efficiently and more effectively than if the anchor member 12 were loaded pre-integration. For example, in the second surgical procedure, a load may be applied to the vertebral element V through the integrated anchor member 12, the inserted attachment member 14, and a spinal implant such as a rod R shown in FIG. 3. The loading may be a compression load or a distraction load to adjust vertebral spacing. The loading may also be a lateral load in an attempt to bring the patient's spine into proper alignment. In either case, the loading may be advantageously maintained with the attached rod R so that the desired surgical result can be achieved.

Various conditions may be employed to determine when or if integration has been achieved for performance of the second surgical procedure. Such techniques include, for example, awaiting the passage of a certain period of time, which can be based on known integration rates, experience, or anatomical studies. For example, the passage of time may extend from a period of a few weeks to several months before the second surgical procedure is performed. Integration of the loading members can also be based in whole or in part on the evaluation of radiographic, fluoroscopic or other imaging information taken of the loading members in situ. The second surgical procedure may be performed once any of these conditions are satisfied.

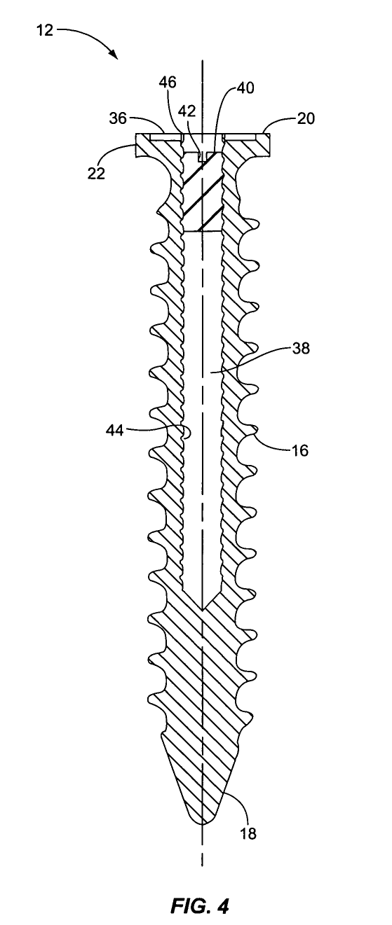

Referring to FIGS. 2 and 3, the exemplary attachment member 14 comprises an attachment stem 32 that is insertable into the anchor member 12. In one embodiment, the attachment stem 32 comprises threads 33 that match corresponding threads 44 (not specifically shown in FIGS. 1-3, but see FIG. 4) in the anchor member 12. Alternative embodiments may comprise other means for coupling or engaging the attachment member 14 and anchor member 12, including, for example, twist-lock fastening, friction locking, interference fitting, adhesive locking, push-pin fastening, spring fastening, or other retaining mechanisms known in the art. In an embodiment comprising threads, the attachment member 14 may be coupled to the anchor member 12 by screwing the attachment member 14 into the anchor member 12 to a working height as shown in FIG. 3. The working height may vary by application, but generally represents a height where an enlarged head portion 30 of the attachment member 14 is brought into close proximity with or adjacent to the proximal end 20 of the anchor member 12. The head portion 30 may also be placed in contact with the anchor member 14. The working height for the attachment member permits placement of a spinal implant such as the rod R into the attachment member 14 so that the rod R is supported by the head portion 30 and between axially extending arms 26, 28. A conventional retainer, such as a pin, nut, or setscrew 32 may then be used to secure the rod R to the attachment member 14. Other spinal implant securing mechanisms may be used and are discussed in greater detail below.

Note that the working height does not expressly require that the attachment member 14 be tightened down on anchor member 12. Some gap may remain between the head portion 30 and the anchor member 12 or vertebral member V. In fact, proper alignment of the attachment member 14 to a spinal implant such as a rod R may preclude the attachment member 14 from being completely tightened. In some instances, some locking feature, such as elastomeric Nylon® threads (not specifically shown), may be incorporated into one or both of the attachment member 14 and anchor member 12 to retain the relative position between the two components.

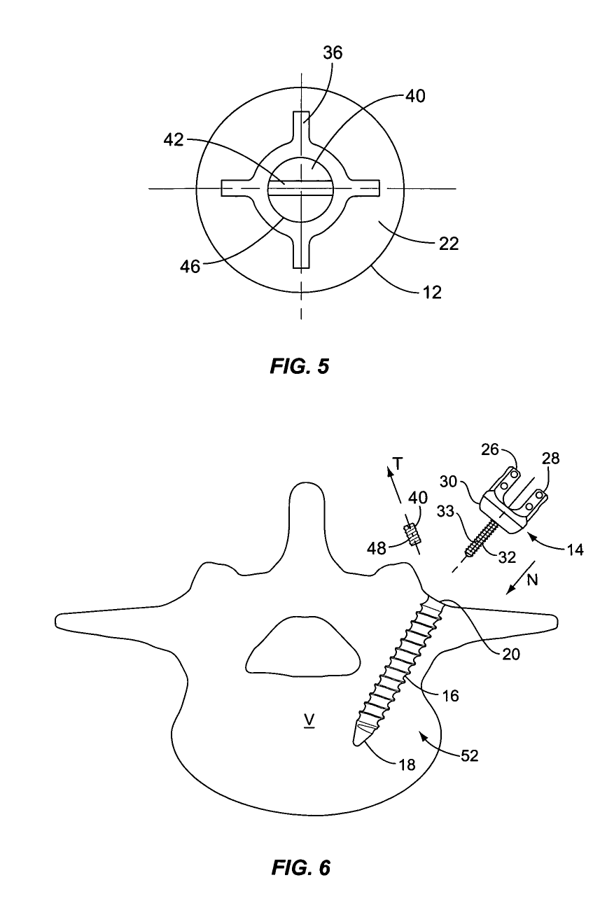

FIG. 4 shows a side cross section view of an exemplary anchor member 12. As discussed above, the anchor member 12 comprises a distal end 18 and a proximal end 20 with an externally threaded engagement portion 16 therebetween. An enlarged flange 22 may be disposed at or near the proximal end 20. The proximal end 20 further comprises a drive feature 36 to screw the anchor member 12 to the desired position within a vertebral member V. In one embodiment, the drive feature 36 is a cross-drive feature as shown in FIG. 5, although other drive types may be used. For example, some conventionally known alternatives include slotted, star, spline, square, triple square, and internal or external hex drivers.

FIG. 4 also shows a hollow interior cavity 38 within the body of the anchor member 12. The cavity 38 extends from an opening 46, axially along the length of the anchor member 12. The size of the cavity may be configured to correspond to the size of stem portion of 32 of attachment member 14 (see FIGS. 1 and 2). In general, the walls of cavity 38 are sized to correspond to that of stem portion 32 of attachment member 14 so that there is little or no interference between the stem portion 32 and cavity 38. Thus, inserting the stem portion 32 of attachment member 14 into the cavity 38 of the anchor portion 12 does not produce an appreciable outward deflection of the engagement portion 16 of the anchor member 12. Maintaining the size and position of engagement portion 16 within a vertebral member V may advantageously retain the integration obtained between the vertebral member V and the anchor portion 12 that is achieved between surgical procedures. In one embodiment, the walls of the cavity 38 are sized to be greater than or equal to the size of the stem portion 32 of attachment member 14. In one embodiment, the walls of the cavity 38 are threaded 44 to match corresponding threads 33 on the stem portion 32 of the attachment member 14.

The exemplary anchor member 12 shown in FIGS. 4 and 5 also includes a removable plug 40 inserted near the opening 46 of cavity 38. It is contemplated that the anchor member 12 may be left for a period of time to integrate within a vertebral member V. Therefore, it is desirable to keep the internal cavity 38 as clear as possible to permit subsequent insertion of the attachment member 14. The removable plug 40 therefore operates as a seal to prevent seepage of bodily fluids or bone growth into the internal cavity 38. The removable plug 40 may be constructed of a material that is somewhat pliable (at least relative to anchor member 12) to promote conformity to the substantially rigid anchor member 12. In fact, some amount of interference between the removable plug 40 and the anchor member 12 may be desirable. Exemplary material choices for the removable plug 40 may include biocompatible plastics, monomers, and polymers.

The removable plug 40 shown in FIGS. 4 and 5 also includes its own drive feature 42 to insert and remove the plug 40 from the anchor member 12. In the exemplary embodiment shown, a slotted drive feature 42 is shown, although the previously mentioned drive features (e.g., cross, star, spline, square, triple square, and hex) are equally applicable here. In one embodiment, the removable plug 40 has external threads 48, shown in FIG. 6, corresponding to matching threads 44 in cavity 38 of anchor member 12. Consequently, the drive feature 42 may be used to screw the removable plug 40 into the cavity 38 of anchor member 12. The drive feature 42 may also be used to unscrew the removable plug 40 out of the anchor member 12 as indicated by the arrow labeled T. Once the removable plug 40 is removed, such as during a second surgical procedure following the integration of anchor member 12, the attachment member 14 may be inserted into the anchor member 12 by screwing the stem portion 32 into the cavity 38 of anchor member 12 (indicated by arrow N).

FIG. 6 also shows a slight modification to the anchor member 12 inserted in vertebral member V. In this alternative embodiment, the exemplary anchor member 52 does not have the aforementioned flange 22 shown on anchor member 12 in FIGS. 1-5. The absence of a flange permits insertion of the anchor member 52 to a variety of depths within the vertebral member V, including to a depth slightly below the surface of vertebral member V. Consequently, as attachment member 14 is inserted into the anchor member 52, the attachment member 14 may be placed in physical contact with the vertebral member V. This is particularly true if the anchor member 52 in inserted below the surface of the vertebral member V.

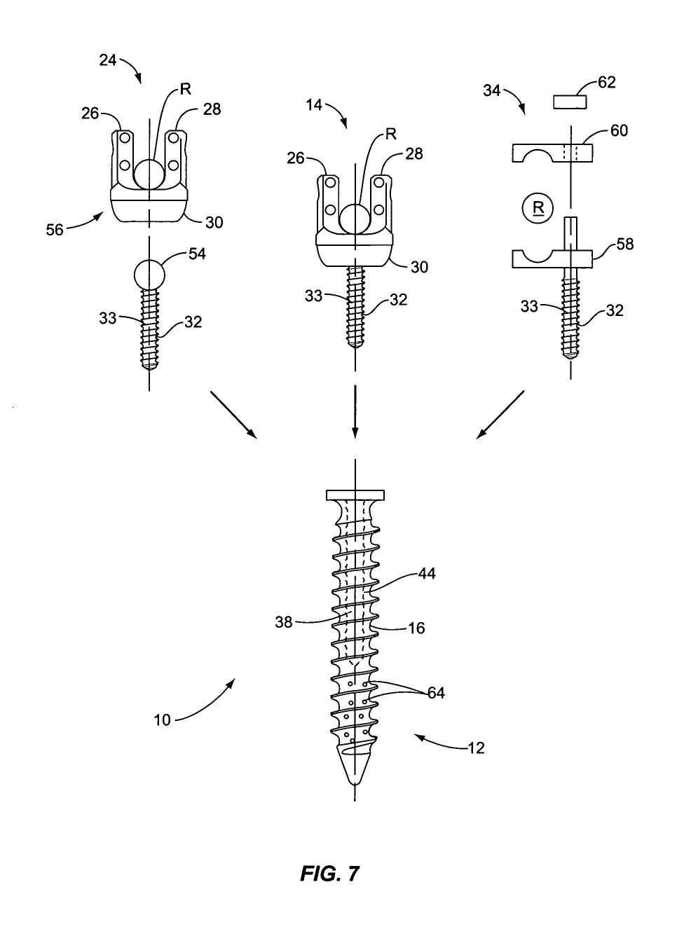

To now, a single attachment member 14 has been discussed in conjunction with the exemplary anchor members 12, 52. FIG. 7 shows that a plurality of attachment members 14, 24, 34, each having a different spinal implant attachment mechanism, may be used in the vertebral attachment device 10. In fact, FIG. 7 shows only three possible configurations. Other spinal implant attachment mechanisms known in the art may also be used as part of the multi-piece vertebral attachment device 10.

In each of the exemplary embodiments shown in FIG. 7, the attachment member 14, 24, 34 has a stem portion 32. Each stem portion 32 may have the aforementioned threads 33 corresponding to internal threads 44 in the internal cavity 38 of anchor member 12. Exemplary attachment member 24 is comprised of multiple components, including a detachable rod support 56 having a base 30 and arms 26, 28 similar to attachment member 14. In addition, a spherical mount 54 permits multi-axial positioning of the rod support 56 once coupled to the mount 54. Thus, the spherical mount 54 also permits multi-axial positioning of a rod R relative to the anchor portion 12 and, hence, vertebral member V. The attachment members 14, 24, 34 are preferably constructed of a non-resorbable, biocompatible material, such as carbon-reinforced polymer composites, shape-memory alloys, titanium, titanium alloys, cobalt chrome alloys, stainless steel, ceramics and combinations thereof.

The exemplary attachment member 34 is similarly comprised of multiple components and permits offset mounting of a rod R relative to stem portion 32 and anchor member 12. The exemplary attachment member 34 is comprised of opposed plates 58, 60 that may be secured to clamp a rod R in place relative to the stem portion 32 and anchor member 12 using a fastener 62 such as a nut, pin, rivet, or screw. As indicated, the exemplary attachment members 14, 24, 34 shown in FIG. 7 are intended merely to be non-limiting representations of the types of attachment members that may be used in the attachment device 10. In general, the attachment member may be any device configured to attach by engaging, retaining, clamping, fastening, holding, contacting, securing or otherwise maintaining the spinal implant to the respective anchor member 12, 52.

FIG. 7 also shows that anchor member 12 may include bone integration features 64 along at least a part of engagement portion 16. Such bone integration features 64 may include, for example, holes, pores, one or more receptacles, one or more chambers or channels, a porous coating, or exterior surface features. The integration features 64 should allow bone to at least partially grow into, adhere to, attach, resorb and/or form with the engagement portion 16 to integrate anchor member 12 to the bony and/or soft tissue structure of the respective vertebral elements V. It may also be desirable in certain instances to install the anchor member 12 into a vertebral member 14 using a conventional adhesive, such as polymethyl-methacrylate or methylmethacrylate. In other embodiments, bone growth stimulators, bone morphogenetic proteins (BMP), or genetic growth factors may be used to promote bone growth around and into the anchor member 12 to improve integration. One example of a BMP suitable for this application is Infuse® available from Medtronic Sofamor Danek in Memphis, Tenn., USA. Each of these optional features may be included to promote integration of the anchor member 12 into a vertebral member V.

The ability to use different attachment members 14, 24, 34 with a single anchor member 12 may be particularly helpful in revision surgeries or in corrective surgeries that are performed in multiple stages. For example, in the correction of certain degenerative conditions such as scoliosis, incremental corrections may be indicated to permit gradual correction of the condition and reduce patient stress. The modular nature of the attachment device 10 may advantageously permit replacement of one attachment member 14, 24, 34 with another during subsequent surgical procedures. Further, the anchor member 12 may advantageously provide a consistent load bearing interface to vertebral members V not otherwise possible where conventional vertebral screws are removed and replaced. With the present attachment device 10, the integration between the anchor member 12 and the vertebral member V is not disrupted by the removal and installation of the attachment members 14, 24, 34.

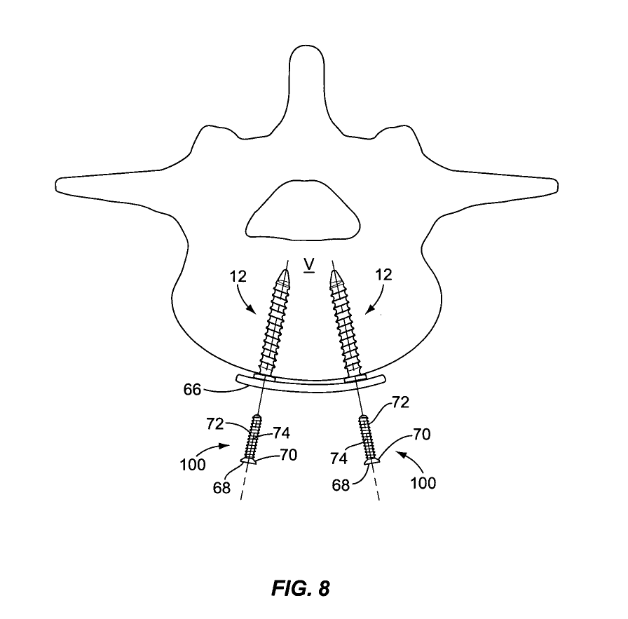

The previous embodiments of the attachment device 10 have represented pedicle screw implementations. Other vertebral attachment points are also contemplated as shown in FIG. 8. FIG. 8 shows an attachment device 100 adapted for use with an anterior plate 66. Laterally installed plates are certainly also applicable. Plate 66, often used in cervical or lumbar regions of the spine, may be used in aligning or fusing adjacent vertebral members V. An anchor member 12 is inserted into the vertebral member V as discussed above. That is, the anchor member 12 may be installed during a first procedure, with the plate 66 and screw members 68 being installed during a subsequent procedure. The exemplary screw members 68 have an enlarged head 70 that may include a conical or spherical seat to permit multi-axial installation. In addition, screw members 68 have a stem portion 72 that is sized to fit within the inner cavity 38 of anchor members 12. In one embodiment, the stem portion 72 is threaded 74 to match internal threads 44 in the cavity 38 of anchor member 12. As before, the multi-piece nature of the attachment device 100 permits secure integration of the anchor member 12 prior to loading the screw-to-vertebra interface with plate 66 and screw members 68.

The present invention may be carried out in other specific ways than those herein set forth without departing from the scope and essential characteristics of the invention. For example, while the various embodiments have been described in conjunction with rod and plate spinal implants, other vertebral constructs may be used to correct and support spinal conditions. For instance, systems using hooks, staples, cables and other devices requiring secure anchoring to a vertebral element may use the teachings disclosed herein. The present embodiments are, therefore, to be considered in all respects as illustrative and not restrictive, and all changes coming within the meaning and equivalency range of the appended claims are intended to be embraced therein.

What is claimed is:

1. A device to attach to a vertebral member comprising:
a first member having a fixed outer diameter and a threaded exterior surface adapted to engage said vertebral member, the first member further having a hollow interior and further having an engagement mechanism, the hollow interior at least partly positioned within the vertebral member when the threaded exterior surface is engaged with said vertebral member; and
a second member having an outer diameter sized to fit within the hollow interior of the first member and engage with the engagement mechanism, the second member being sized to prevent the first member from expanding during insertion of the second member.

2. The device of claim 1 wherein the second member further comprises threads adapted to mate with a threaded interior surface of the first member.

3. The device of claim 1 wherein the second member further comprises an enlarged head portion that limits the insertion depth of the second member into the first member.

4. The device of claim 1 wherein the second member further comprises an attachment portion adapted to couple to a spinal implant device.

5. The device of claim 4 wherein the attachment portion is multi-axially adjustable.

6. The device of claim 1 wherein the first member comprises a proximal end and a distal end, the distal end being closed.

7. The device of claim 1 further comprising a removable plug sized to fit within the hollow interior of the first member.

8. The device of claim 7 wherein the removable plug is threaded to mate with the engagement mechanism of the first member.

9. The device of claim 1 wherein the first member has a substantially conical shape.

10. A device to attach to a vertebral member comprising:
a first member having exterior threads and a hollow interior disposed internal to the exterior threads, the exterior threads adapted to engage said vertebral member, the first member further having a hollow interior;
a second member sized to fit into the hollow interior of the first member; and
a removable plug sized to fit within and seal the hollow interior.

11. The device of claim 10 wherein the hollow interior of the first member comprises internal threads.

12. The device of claim 11 wherein the second member further comprises external threads adapted to engage the internal threads of the first member.

13. The device of claim 11 wherein the removable plug further comprises external threads adapted to engage the internal threads of the first member.

14. The device of claim 10 wherein the second member comprises a coupling to attach to a spinal implant device.

15. The device of claim 14 wherein the coupling is multi-axially adjustable.

16. The device of claim 10 wherein the removable plug further comprises a drive feature to insert and remove the removable plug from the first member.

17. The device of claim 10 wherein the first member is constructed of a biocompatible metal.

18. A method of attaching a device to a vertebral member, the method comprising:
during a first surgical procedure, screwing a first member into a vertebral member with a distal end positioned within the vertebral member and a proximal end adjacent to a surface of the vertebral member;
waiting until a predetermined condition is satisfied for the first member to become set within the vertebral member; and
during a second surgical procedure after the predetermined condition is satisfied, positioning a second member into the first member at a depth within the vertebral member to bring a head portion of the second member to a working height adjacent to the proximal end of the first member.

19. The method of claim 18 wherein the step of positioning the second member into the first member further comprises maintaining a fixed outer diameter of the first member.

20. The method of claim 18 further comprising attaching a spinal implant device to the second member.

21. The method of claim 18 further comprising removing a removable plug from interior threads of the first member prior to positioning the second member into the first member.

22. The method of claim 18 wherein the step of screwing the first member into the vertebral member further comprises applying an adhesive to integrate the first member to the vertebral member.

23. The method of claim 18 wherein the step of screwing the first member into the vertebral member further comprises applying a bone morphogenetic protein to integrate the first member to the vertebral member.

24. The method of claim 18 further comprising:
during a third surgical procedure distanced temporally from the second surgical procedure, unscrewing the second member from the first member and screwing a third member into interior threads of the first member to a depth within the vertebral member and to bring a head portion of the third member to a working height adjacent to the proximal end of the first member.

25. The method of claim 24 further comprising attaching a spinal implant device to the third member.

26. A method of attaching a device to a vertebral member comprising the steps of:
during a first procedure, inserting a first member into a vertebral member, the first member having a removable plug to seal a hollow interior;
waiting until a predetermined condition is satisfied for the first member to become set within the vertebral member; and
during a second procedure after the predetermined condition is satisfied, removing the removable plug from the first member and inserting a second member into the hollow interior of the first member.

27. The method of claim 26 further comprising coupling a spinal implant device to the second member.

28. The method of claim 26 wherein the step of inserting the first member into the vertebral member comprises screwing the first member into the vertebral member.

29. The method of claim 26 wherein the step of removing the removable plug from the first member comprises unscrewing the removable plug from the first member.

30. The method of claim 26 wherein the step of inserting a second member into the hollow interior of the first member comprises screwing the second member into the hollow interior of the first member.

31. The method of claim 26 wherein the step of inserting the first member into a vertebral member comprises applying an adhesive to the first member to promote integration of the first member to the vertebral member.

32. The method of claim 26 wherein the step of inserting the first member into a vertebral member comprises applying a bone morphogenetic protein to the first member to promote integration of the first member to the vertebral member.
